(19) United States
(12) Patent Application Publication
(10) Pub. No.: US 2003/0105349 A1
(43) Pub. Date: Jun. 5, 2003

(54) Preparation of beta-ketonitriles

(76) Inventor: Oskar Koch, Gottingen (DE)

Correspondence Address: BAYER POLYMERS LLC, 100 BAYER ROAD, PITTSBURGH, PA, 15205, US

(21) Appl. No.: 10/305,861

(22) Filed: Nov. 27, 2002

(30) Foreign Application Priority Data
Dec. 3, 2001 (DE) 10159329.5

(51) Int. Cl. C07C 253/30

(52) U.S. Cl. 558/357

(57) ABSTRACT

The present invention relates to a process for preparing β-ketonitriles.

### FIELD OF THE INVENTION

The present invention relates to a process for preparing β-ketonitriles.

### BACKGROUND OF THE INVENTION

β-Ketonitriles are important intermediates for preparing active pharmaceutical (WO-A 99/23091) and agrochemical ingredients (EP-A 496 630).

EP-A 220 220 describes the preparation by condensation of carboxylic esters in an excess of acetonitrile with the use of sodium methoxide. The methanol resulting from the reaction is continuously distilled off together with acetonitrile which results in a yield of 83% of theory, based on the carboxylic ester used in deficiency. However, this process has the disadvantage that a 13-14-fold excess of acetonitrile is used, which is undesirable from an occupational hygiene point of view.

Although the reaction of carboxylic esters with acetonitrile under sodium ethoxide catalysis in a stoichiometric ratio is described in J. Am. Chem. Soc. 56, 1172 (1934), moderate yields of only 44% of theory result.

Accordingly, it is an object of the present invention to find a process which avoids an excess of acetonitrile and makes good yields possible.

The avoidance of an excess of acetonitrile, which is known to have very good dissolving properties as a polar aprotic solvent, leads to process engineering problems and to a deterioration in the yield of the desired β-ketonitriles. For example, the avoidance of an acetonitrile excess may lead to a reaction mixture which is difficult to stir (porridging). This porridging is problematic in particular when the β-ketonitrile-containing reaction mixture is cooled to low temperatures, for example room temperature, before hydrolysis, in order to avoid reducing the yield of the hydrolysis-sensitive β-ketonitriles.

### SUMMARY OF THE INVENTION

We have found that the underlying object of this invention is achieved, surprisingly, by an excess of carboxylic ester, based on the quantity of acetonitrile.

Accordingly, the invention accordingly provides a process for preparing β-ketonitriles of the formula (I)

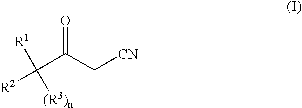

where

n=0 or 1 and, when n=1,

R¹=H or methyl

R² and R³ are each independently methyl or ethyl or

R² and R³ together are an optionally substituted 3- to 6-membered ring

or when n=0

R¹ and R² together are an optionally substituted 3- to 6-membered ring,

by reacting acetonitrile with carboxylic esters of the formula (II)

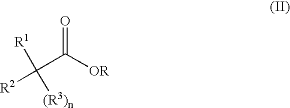

where

n and also

R¹ to R³ are as defined above

R is a C₁- to C₄-alkyl radical,

in the presence of an alkali metal alkoxide, wherein the molar ratio of carboxylic ester (II) to acetonitrile is in the range from 2:1 to 10:1.

When R² and R³ together are a 3- to 6-membered ring, preference is given to cyclopropyl, cyclopentyl and cyclohexyl. The rings may additionally contain a double bond or optionally two double bonds. The rings may also optionally contain heteroatoms such as N, S or O.

When R¹ and R² together are an optionally substituted 3- to 6-membered ring, preference is given to aromatic radicals, optionally having one or two heteroatoms in the ring. More preference is given to phenyl radicals. In substituted phenyl radicals, preference is given to lower alkyl radical or lower alkoxy radical substituents, in particular the methoxy or methyl substituents. Preference is given to a substituent in the para position.

Preference is given to preparing 4-methyl-3-oxovaleronitrile, 4,4-di-methyl-3-oxovaleronitrile, 3-cyclopropyl-3-oxopropionitrile, 3-cyclopentyl-3-oxopropionitrile, 3-cyclohexyl-3-oxopropionitrile, 3-phenyl-3-oxopropionitrile, 3-(p-methoxyphenyl)-3-oxopropionitrile and 3-(p-methylphenyl)-3-oxopropionitrile, and greater preference to 4-methyl-3-oxovaleronitrile, 4,4-dimethyl-3-oxovaleronitrile, 3-cyclopropyl-3-oxopropionitrile and 3-phenyl-3-oxopropionitrile.

Preference is given to R=methyl (Me) or ethyl (Et), and more preference is given to R=methyl.

The carboxylic esters are generally used in a 2- to 10-fold molar, preferably in a 3- to 5-fold molar, excess, based on acetonitrile.

The molar ratio of alkali metal alkoxide to acetonitrile is preferably in the range from 0.8 to 1.4:1, more preferably in the range from 1.0 to 1.2:1.

Preferred alkali metal alkoxides include KOMe, NaOMe, KOEt and NaOEt. These alkali metal alkoxides may also be used as mixtures. More preference is given to sodium methoxide.

The reaction may be carried out in the presence of an inert solvent, but the reaction is preferably conducted without solvent.

The process is carried out at temperatures which are in the range of the boiling points of the alcohols resulting from the reaction, typically from 60 to 120° C. or else at temperatures which result from the formation of azeotropic mixtures between these alcohols and acetonitrile (for example methanol/acetonitrile).

The process is advantageously operated while distilling off the alcohol ROH formed during the reaction. The reaction is generally ended after 4 to 5 hours.

Hydrolysis is also possible at temperatures distinctly higher than room temperature, without noticeable yield losses having to be accepted. For instance, this particular embodiment allows yields of 85% of theory to be achieved, based on the conversion of the acetonitrile used in deficiency.

Preference is thus given to carrying out the work-up by hydrolysis in such a manner that the reaction mixture at a temperature of 60 to 90° C., preferably 75 to 80° C., is rapidly introduced into water with stirring. The temperature of the water is typically in the range from 0 to 30° C.

After hydrolysis, the product is present as the alkali metal enolate in the aqueous phase and is liberated as the ketone by acidifying with a mineral acid (for example hydrochloric or sulfuric acid) and then extracted with a water-immiscible organic solvent. Any purification may be effected by distillation or crystallization.

The excess carboxylic acid separates after hydrolysis as the upper organic phase and can be reused in the reaction after removal and drying. This drying is advantageously carried out by adding a volatile hydrocarbon (for example heptane, cyclohexane) which forms an azeotrope with water by distillatively freeing the carboxylic acid from the water with the aid of the azeotropic mixture.

The work-up may also be effected in such a manner that the mixture is acidified after hydrolysis and the excess carboxylic ester together with the product combine as the organic phase. The use of an additional solvent is then unnecessary.

The invention is further illustrated but is not intended to be limited by the following examples in which all parts and percentages are by weight unless otherwise specified.

### EXAMPLES

### Example 1

4,4-Dimethyl-3-oxovaleronitrile

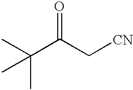

812 g (7.0 mol) of methyl pivalate, 82 g (2.0 mol) of acetonitrile and 119 g (2.2 mol) of sodium methoxide were introduced into a 2 l jacketed vessel having a bottom discharge valve and heated to 88-90° C. for 1.5 h with stirring. About 160 g of a mixture of methanol and acetonitrile which contained about 13% by weight of acetonitrile were then distilled off at a top temperature of 70 to 75° C. for 3 h. After the temperature of the reaction mixture was reduced to 80° C., this was introduced into 500 g of water (water temperature 20° C.) with stirring. After phase separation, 441 g of methyl pivalate were obtained, the aqueous phase adjusted to pH 5 using 220 g of semiconcentrated sulfuric acid and the product extracted using 200 g of xylene. After purification by distillation, 160 g of 4,4-dimethyl-3-oxovaleronitrile were obtained, which corresponded to a yield of 86% of theory, based on the conversion of acetonitrile.

Alternatively, the work-up may be effected by acidifying to pH 6 to 7 after hydrolysis without adding a solvent (for example xylene) and accordingly separating the product and the recovered methyl pivalate together as the organic phase. Subsequent distillation leads to the same yield result.

### Example 2

4-Methyl-3-oxovaleronitrile

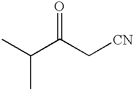

Example 1 is repeated using corresponding quantities of ethyl isobutyrate, acetonitrile and sodium ethoxide. The yield of 4-methyl-3-oxovaleronitrile was 84% of theory, based on the conversion of acetonitrile.

### Example 3

3-Cyclopropyl-3-oxopropionitrile

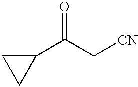

Example 1 was repeated using corresponding quantities of methyl cyclopropanecarboxylate, acetonitrile and sodium ethoxide. The yield of 3-cyclopropyl-3-oxopropionitrile was 82% of theory, based on the conversion of acetonitrile.

### Example 4

3-Phenyl-3-oxopropionitrile

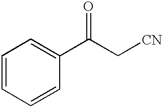

Example 1 was repeated using corresponding quantities of methyl benzoate, acetonitrile and sodium ethoxide. The yield of 3-phenyl-3-oxopropionitrile was 85% of theory, based on the conversion of acetonitrile.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for preparing β-ketonitriles of the formula (I) 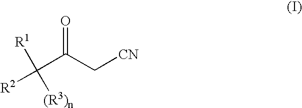
wherein
n=0 or 1 and, when n=1,
R¹=H or methyl
R² and R³ are each independently methyl or ethyl or
R² and R³ together are an optionally substituted 3- to 6-membered ring
or when n=0
R¹ and R² together are an optionally substituted 3- to 6-membered ring,
comprising the step of reacting acetonitrile with carboxylic esters of the formula (II) 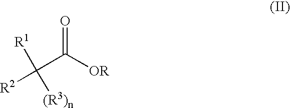
wherein
n and also
R¹ to R³ are as defined above and
R is a C₁- to C₄-alkyl radical,
in the presence of an alkali metal alkoxide, wherein the molar ratio of carboxylic ester (II) to acetonitrile is in the range from 2:1 to 10:1.

2. A process according to claim 1, wherein the alkali metal alkoxide is sodium methoxide, sodium ethoxide, potassium methoxide or potassium ethoxide or a mixture thereof.

3. A process according to claim 1, wherein no solvent is added in the reaction of acetonitrile and carboxylic ester (II).

4. A process according to claim 1, wherein alcohol formed is distilled out of the reaction of acetonitrile and carboxylic ester (II).

5. A process according to claim 1, wherein the reaction of acetonitrile and carboxylic ester (II) is hydrolyzed at a temperature of 60-90° C.
